(19) United States
(12) Patent Application Publication
(10) Pub. No.: US 2007/0041911 A1
(43) Pub. Date: Feb. 22, 2007

(54) HFC SOLUTION FORMULATIONS CONTAINING SALBUTAMOL HYDROCHLORIDE OR SALBUTAMOL CITRATE

(76) Inventors: Friedrich Schmidt, Ingelheim am Rhein (DE); Mariola Mann, Bingen (DE); Michael Cope, Woking Surrey (GB); Holger Memmesheimer, Ockenheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/463,477

(22) Filed: Aug. 9, 2006

(30) Foreign Application Priority Data
Aug. 12, 2005 (EP) 05017599

(51) Int. Cl. A61L 9/04; A61K 31/573; A61K 31/137; A61K 31/4745

(52) U.S. Cl. 424/45; 514/649; 514/291; 514/171

(57) ABSTRACT

This invention relates to stabilized aerosol solution formulations containing acid addition salts of salbutamol, which are suitable for administration by metered dose inhalers (MDIs). More particularly, this invention relates to solution formulations containing one of the aforementioned salts of salbutamol, preferably salbutamol hydrochloride or salbutamol citrate, more preferably one of the two before mentioned salts in combination with one or more additional pharmacologically active substances, together with an environmentally safe hydrofluorocarbon (HFC) as a propellant, a co-solvent, preferably an organic compound as a co-solvent, and either an inorganic acid or an organic acid.

This invention relates to stabilized aerosol solution formulations containing acid addition salts of salbutamol, which are suitable for administration by metered dose inhalers (MDIs). More particularly, this invention relates to solution formulations containing one of the aforementioned salts of salbutamol, preferably salbutamol hydrochloride or salbutamol citrate, more preferably one of the two before mentioned salts in combination with one or more additional pharmacologically active substances, together with an environmentally safe hydrofluorocarbon (HFC) as a propellant, a co-solvent, preferably an organic compound as a co-solvent, and either an inorganic acid or an organic acid.

### BACKGROUND OF THE INVENTION

Salbutamol is a highly effective β-sympathomimetic agent, which can be used to treat respiratory complaints, particularly COPD (chronic obstructive pulmonary disease) and asthma. The term salbutamol refers to the free amine base. It has the following chemical structure:

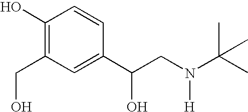

Salbutamol and its sulphate salt have been used as pharmacologically active drug substances for a long time. They are comprehensively described in the relevant literature, e.g. the European Pharmacopoeia.

For treating the abovementioned complaints, it is useful to administer the active substance by inhalation. In addition to the administration of broncholytically active compounds in the form of inhalable powders containing the active substance the administration can also occur in form of hydrofluorocarbon containing aerosol formulations.

The administration of aerosol formulations by means of pressurized, metered-dose inhalers (MDIs) is used widely in therapy, such as in the treatment of obstructive airway diseases and asthma. Compared with oral administration, inhalation provides more rapid onset of action while minimizing systemic side effects. Aerosol formulations can be administered by inhalation through the mouth or topically by application to the nasal mucosa.

Formulations for aerosol administration via pressurized MDIs can be solutions or suspensions. Solution formulations offer the advantage of being homogeneous in nature with the medicament(s) and excipient(s) completely dissolved in the propellant vehicle. Solution formulations also obviate physical stability problems associated with suspension formulations and thus assure more consistent uniform dosage administration while also eliminating the need for surfactants.

The administration of aerosol solution formulations via pressurized MDIs is dependent upon the propulsive force of the propellant system used in its manufacture. Traditionally, the propellant comprised a mixture of chlorofluorocarbons (CFCs) to provide the desired solubility, vapor pressure, and stability of the formulation. However, since it has been established in recent years that CFCs are environmentally of disadvantage because they contribute to the depletion of the Earth's ozone layer, it is desirable to substitute environmentally safe hydrofluorocarbon (HFC) propellants or other non-chlorinated propellants for environmentally harmful CFC propellants in aerosol inhalation formulations.

For example, U.S. Pat. No. 4,174,295 discloses the use of propellant systems consisting of combinations of HFCs, which may also contain a saturated hydrocarbon component, suitable for application in the fields of home products such as hair lacquers, anti-perspiration products, perfumes, deodorants, paints, insecticides and the like.

It is known in the art that certain HFCs have properties suitable for use as propellants for the aerosol administration of medicaments. For example, published European patent Application No. 0 372 777 (EP 089312270.5) describes the use of 1,1,1,2-tetrafluoroethane (HFC-134(a)) in combination with at least one “adjuvant” (a compound having a higher polarity than the HFC-134(a)) and a surface active agent to prepare suspension and solution formulations of medicaments suitable for administration by the aerosol route.

Also, PCT Published Application No. W0 91/11496 (PCT/EP91/00178) discloses the use of 1,1,1,2,3,3,3-heptafluoropropane (HFC-227), optionally mixed with other propellant components, for use in preparing suspension aerosol formulations of medicaments. U.S. Pat. No. 2,868,641 and U.S. Pat. No. 3,282,781 disclose aerosol compositions comprising a medicament (epinephrine or isoproterenol HCl), a co-solvent, a propellant and ascorbic acid as anti-oxidant. European Patent EP 673 240 B1 proposes the addition of acids to medicinal aerosol formulations in order to provide for the stabilization of the medicament.

US application 2002/0002204 describes stable packed aqueous formulations of albuterol or pharmaceutically acceptable salts thereof.

However, none of the aforesaid mentioned compositions or formulations of US 2002/0002204 are described for its use in pressurized MDIs. It is known by the person skilled in the art that salts in MDI solution formulations for pharmaceutical use are difficult to handle due to the lack of good solubility. Especially there is no disclosure of special salbutamol salts in aerosol solution formulations for their use in pressurized MDIs.

### DESCRIPTION OF THE INVENTION

The present invention provides stabilized aerosol solution formulations comprising a salbutamol acid addition salt, and potentially one or several additional pharmacologically active substances, an HFC propellant, a co-solvent, and an inorganic or an organic acid. The formulations are suitable for administration in pressurized MDIs, which contain multiple doses of a formulation.

The term “aerosol solution formulation” means a pharmaceutical formulation of a medicament suitable for aerosol administration wherein the medicament and excipients are completely dissolved.

The term “stabilized aerosol solution formulation” means an aerosol solution formulation which exhibits substantial chemical stability over time. The acid in the formulation provides stability by interaction of the medicament with the co-solvent and/or water present in the solution formulation.

The aerosol solution formulation according to the invention preferably contains 0.001 to 1.0%, preferably 0.01 to 0.5%, more preferably 0.05 to 0.3% salbutamol. The concentrations are referred to the free salbutamol base.

Amongst the acid addition salts with pharmacologically acceptable acids, which may be formed, are the followings salts consisting from the group of hydrochloride, hydrobromide, hydroiodide, (hydro)phosphate, (hydro)methansulfonate, (hydro)nitrate, (hydro)maleate, (hydro)acetate, (hydro)benzoate, (hydro)citrate, (hydro)fumarate, (hydro)tartrae, (hydro)oxalate, (hydro)succinate, and (hydro)-p-toluolsulfonate.

The salbutamol acid addition salt in the formulation according to the invention is preferably either the hydrochloride or the citrate, which is the addition product of salbutamol and hydrochloric acid, resp. of salbutamol and citric acid.

The addition product of salbutamol and citric acid can be synthesized by the conversion of citric acid with salbutamol base. The resulting product consists of one or more of the following compounds of formula Ia, Ib and/or Ic: Compound of formula Ia, 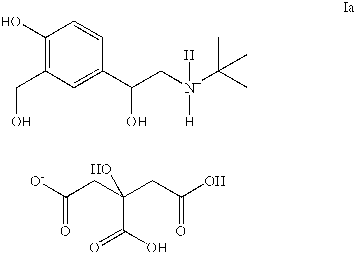
which is the mono-salbutamol citrate.

Compound of formula Ib, 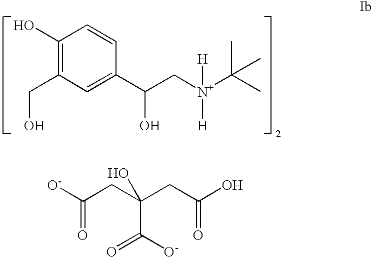
which is the di-salbutamol citrate.

Compound of formula Ic, 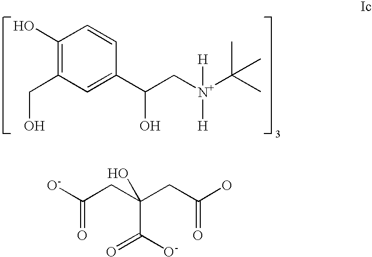
which is the tri-salbutamol citrate.

The resulting product of the reaction of salbutamol base with hydrochloric acid is the compound of formula Ia, 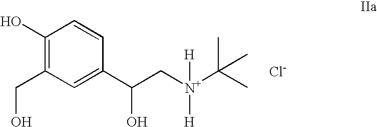
which is salbutamol hydrochloride.

If salbutamol hydrochloride is used, the aforementioned amounts correspond to 0.00115 to 1.115% salbutamol hydrochloride, preferably 0.0115 to 0.575%, more preferably 0.0575 to 0.345% salbutamol hydrochloride.

If salbutamol citrate is used, the aforementioned amounts correspond to 0.0018 to 1.8% salbutamol citrate, preferably 0.018 to 0.9%, more preferably 0.09 to 0.54% salbutamol citrate.

The formulation according to the invention might contain additional pharmacologically active substances or mixtures of substances, preferably selected from those groups:

Anticholinergics:

Anticholinergics preferably selected from the group consisting of tiotropium, tiotropiumbromide, oxitropiumbromide, flutropiumbromide, ipratropiumbromide, glycopyrroniumsalts, trospiumchloride, tolterodin, 2,2-diphenylpropionacidtropenolester-methobromide, 2,2-diphenylpropionacidscopinester-methobromide, 2-fluoro-2,2-diphenylacidicacidscopinester-methobromide, 2-fluoro-2,2-diphenylacidicacidtropenolester-methobromide, 3,3′,4,4′-tetrafluorbenzilacidtropenolester-methobromide, 3,3′,4,4′-tetrafluorbenzilacidscopinester-methobromide, 4,4′-difluorbenzilacidtropenolester-methobromide, 4,4′-difluorbenzilacidscopinester-methobromide, 3,3′-difluorobenzilacidtropenolester-methobromide, 3,3′-difluorobenzilacidscopinester-methobromide, 9-hydroxy-fluoren-9-carbonacidtropenolester-methobromide, 9-fluoro-fluoren-9-carbonacidtropenolester-methobromide, 9-hydroxy-fluoren-9-carbonacidscopinester-methobromide, 9-fluoro-fluoren-9-carbonacidscopinester-methobromide, 9-methyl-fluoren-9-carbonacidtropenoleste-methobromide, 9-methyl-fluoren-9-carbonacidscopineste-methobromide, benzilacidcyclopropyltropinester-methobromide, 2,2-diphenylpropionacidcyclopropyltropinester-methobromide, 9-hydroxy-xanthen-9-carbonacidcyclopropyltropinester-methobromide, 9-methyl-fluoren-9-carbonacidcyclopropyltropinester-methobromide, 9-methyl-xanthen-9-carbonacidcyclopropyltropinester-methobromide, 9-hydroxy-fluoren-9-carbonacidcyclopropyltropinester-methobromide, 4,4′-difluorbenzilacidmethylestercyclopropyltropinester-methobromide, 9-hydroxy-xanthen-9-carbonacidtropenolester-methobromide, 9-hydroxy-xanthen-9-carbonacidscopinester methobromide, 9-methyl-xanthen-9-carbonacidtropenolester -methobromide, 9-methyl-xanthen-9-carbonacidscopinester-methobromide, 9-ethyl-xanthen-9-carbonacidtropenolester methobromide, 9-difluormethyl-xanthen-9-carbonacidtropenolester-methobromide, 9-hydroxymethyl-xanthen-9-carbonacidscopinester-methobromide, optionally in the form of the racemates, the enantiomers, the diastereomers and optionally the pharmacologically acceptable acid addition salts, solvates and/or the hydrates thereof.

Beta-Sympathomimetics:

Beta-sympathomimetics preferably selected from the group consisting of albuterol, bambuterol, bitolterol, broxaterol, carbuterol, clenbuterol, fenoterol, formoterol, hexoprenaline, ibuterol, indacaterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramide, terbutaline, tolubuterol, CHF-1035, HOKU-81, KUL-1248, 3-(4-{6-[2-hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol, optionally in the form of the racemates, the enantiomers, the diastereomers and optionally the pharmacologically acceptable acid addition salts, solvates and/or the hydrates thereof.

Steroids:

Steroids preferably selected from the group consisting of prednisolone, prednisone, butixocortpropionate, RPR-106541, flunisolide, beclomethasone, triamcinolone, budesonide, fluticasone, mometasone, ciclesonide, rofleponide, ST-126, dexamethasone, 6α,90α-difluoro-17α-[(2-furanylcarbonyl)oxy]-11,β-hydroxy-16α-methyl-3-oxo-androsta-1,4-dien-17,β-carbothionacid (S)-fluoromethylester, 6α,9α-difluoro-11,β-hydroxy-16α-methyl-3-oxo-17α-propionyloxy-androsta-1,4-dien-17β-carbothionacid (S)-(2-oxo-tetrahydro-furan-3S-yl)ester and etiprednol-dichloroacetat (BNP-166), optionally in the form of the racemates, the enantiomers, the diastereomers and optionally the pharmacologically acceptable acid addition salts, solvates and/or the hydrates thereof.

PDEIV-Inhibitors:

PDEIV-inhibitor preferably selected from the group consisting of enprofyllin, theophyllin, roflumilast, ariflo (cilomilast), CP-325,366, BY343, D-4396 (Sch-351591), AWD-12-281 (GW-842470), N-(3,5-Dichloro-1-oxo-pyridin-4-yl)-4-difluoromethoxy-3-cyclopropylmethoxybenzamide, NCS-613, pumafentine, (−)p-[(4αR*,10bS*)-9-ethoxy-1,2,3,4,4a, 10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexan-1-carbonacid], 2-carbomethoxy-4-cyano-4-(3-cyclopropylmethoxy-4-difluoromethoxyphenyl)cyclohexane-1-on, cis[4-cyano-4-(3-cyclopropylmethoxy-4-difluoromethoxyphenyl)cyclohexane-1-ol], (R)-(+)-ethyl[4-(3-cyclopentyloxy-4-methoxyphenyl)pyrrolidin-2-yliden]acetate, (S)-(−)-ethyl[4-(3-cyclopentyloxy-4-methoxyphenyl)pyrrolidin-2-yliden]acetate, CDP840, Bay-198004, D-4418, PD-168787, T-440, T-2585, arofyllin, atizoram, V-11294A, Cl-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridin and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, the enantiomers, the diastereomers and optionally the pharmacologically acceptable acid addition salts, solvates and/or the hydrates thereof.

LTD4-Antagonists:

LTD4-antagonist preferably selected from the group consisting of montelukast, 1-(((R)-(3-(2-(6,7-difluoro-2-quinolinyl)ethenyl)phenyl)-3-(2-(2-hydroxy-2-propyl)phenyl)thio)methylcyclopropan-acidicacid, 1-(((1(R)-3 (3-(2-(2,3-dichlorothieno[3,2-b]pyridin-5-yI)-(E)-ethenyl)phenyl)-3-(2-(1-hydroxy-1-methylethyl)phenyl)propyl)thio)methyl)cyclopropanacidicacid, pranlukast, zafirlukast, [2-[[2-(4-tert-butyl-2-thiazolyl)-5-benzofuranyl]oxymethyl]phenyl]acidicacid, MCC-847 (ZD-3523), MN-001, MEN-91507 (LM-1507), VUF-5078, VUF-K-8707 und L-733321, optionally in the form of the racemates, the enantiomers, the diastereomers and optionally the pharmacologically acceptable acid addition salts, solvates and/or the hydrates thereof.

EGFR-Kinase-Inhibitors:

cetuximab, trastuzumab, ABX-EGF, Mab ICR-62, 4-[(3-Chlor-4-fluorophenyl)amino]-6-{[4-(morpholin-4-yl)-1-oxo-2-buten-1-yl]amino}-7-cyclopropylmethoxy-chinazolin, 4-[(R)-(1-phenyl-ethyl)amino]-6-{[4-(morpholin-4-yl)-1-oxo-2-buten-1-yl]amino}-7-cyclopentyloxy-chinazolin, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofurane-3-yl)oxy}-chinazoline, 4-[(3-chloro-4-fluoro-phenyl)amino]-6-[2-((S)-6-methyl-2-oxo-morpholine-4-yl)-ethoxy]-7-methoxy-chinazolin, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-chinazoline, 4-[(R)-(1-Phenyl-ethyl)amino]-6-({4-[N-(tetrahydropyrane-4-yl)-N-methyl-amino]-1-oxo-2-buten-1-yl}amino)-7-cyclopropylmethoxy-chinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-chinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-chinazolin, 4-[(3-Ethinyl-phenyl)amino]-6,7-bis-(2-methoxy-ethoxy)-chinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-chinoline, 4-[(R)-(1-phenyl-ethyl)amino]-6-{[4-((R)-6-methyl-2-oxo-morpholin-4-yl)-1-oxo-2-buten-1-yl]amino}-7-methoxy-chinazoline, 4-[(3-chloro-4-fluorophenyl)amino]-6-{[4-(morpholine-4-yl)-1-oxo-2-buten-1-yl]amino}-7-[(tetrahydrofurane-2-yl)methoxy]-chinazoline, 4-[(3-ethinyl-phenyl)amino]-6-{[4-(5,5-dimethyl-2-oxo-morpholin-4-yl)-1-oxo-2-buten-1-yl]amino}-chinazoline, 4-[(3-chloro-4-fluoro-phenyl)amino]-6-{2-[4-(2-oxo-morpholine-4-yl)-piperidine-1-yl]-ethoxy}-7-methoxy-chinazoline, 4-[(3-chloro-4-fluoro-phenyl)amino]-6-(trans-4-amino-cyclohexan-1-yloxy)-7-methoxy-chinazoline, 4-[(3-chloro-4-fluoro-phenyl)amino]-6-(trans-4-amino-cyclohexan-methansulfonylamino-cyclohexan-1-yloxy)-7-methoxy-chinazoline, 4-[(3-chloro-4-fluoro-phenyl)amino]-6-(tetrahydropyrane-3-yloxy)-7-methoxy-chinazoline, 4-[(3-chloro-4-fluoro-phenyl)amino]-6-{1-[(morpholine-4-yl)carbonyl]-piperidin-4-yloxy}-7-methoxy-chinazoline, 4-[(3-chloro-4-fluoro-phenyl)amino]-6-(piperidine-3-yloxy)-7-methoxy-chinazoline, 4-[(3-chloro-4-fluoro-phenyl)amino]-6-[1-(2-acetylamino-ethyl)-piperidin-4yloxy]-7-methoxy-chinazoline, 4-[(3-chloro-4-fluoro-phenyl)amino]-6-(tetrahydropyran-4-yloxy)-7-ethoxy-chinazoline, 4-[(3-chloro-4-fluoro-phenyl)amino]-6-{trans-4-[(morpholin-4-yl)carbonylamino]-cyclohexan-1-yloxy}-7-methoxy-chinazoline, 4-[(3-chloro-4-fluoro-phenyl)amino]-6-{1-[(piperidin-1-yl)carbonyl]-piperidin-4-yloxy}-7-methoxy-chinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-chinazoline, 4-[(3-chloro-4-fluoro-phenyl)amino]-6-(trans-4-ethansulfonylamino-cyclohexan-1-yloxy)-7-methoxy-chinazoline, 4-[(3-chloro-4-fluoro-phenyl)amino]-6-(1-methansulfonyl-piperidin-4-yloxy)-7-(2-methoxy-ethoxy)-chinazoline, 4-[(3-chloro-4-fluoro-phenyl)amino]-6-[1-(2-methoxy-acetyl)-piperidin-4-yloxy]-7-(2-methoxy-ethoxy)-chinazoline, 4-[(3-ethinyl-phenyl)amino]-6-(tetrahydropyran-4-yloxy]-7-methoxy-chinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-chinazoline, 4-[(3-chloro-4-fluoro-phenyl)amino]-6-{cis-4-[(morpholin-4-yl)carbonylamino]-cyclohexan-1-yloxy}-7-methoxy-chinazoline, 4-[(3-chloro-4-fluoro-phenyl)amino]-6-{1-[2-(2-oxopyrrolidin-1-yl)ethyl]-piperidin-4-yloxy}-7-methoxy-chinazoline, 4-[(3-ethinyl-phenyl)amino]-6-(1-acetyl-piperidin-4-yloxy)-7-methoxy-chinazoline, 4-[(3-ethinyl-phenyl)amino]-6-(1-methyl-piperidin-4-yloxy)-7-methoxy-chinazoline, 4-[(3-ethinyl-phenyl)amino]-6-(1-methansulfonyl-piperidin-4-yloxy)-7-methoxy-chinazolin, 4-[(3-chloro-4-fluoro-phenyl)amino]-6-(1-methyl-piperidin-4-yloxy)-7(2-methoxy-ethoxy)-chinazoline, 4-[(3-ethinyl-phenyl)amino]-6-{1-[(morpholin-4-yl)carbonyl]-piperidin-4-yloxy}-7-methoxy-chinazoline, 4-[(3-chloro-4-fluoro-phenyl)amino]-6-{1-[(N-methyl-N-2-methoxyethyl-amino)carbonyl]-piperidin-4-yloxy}-7-methoxy-chinazoline, 4-[(3-chloro-4-fluoro-phenyl)amino]-6-(1-ethyl-piperidin-4-yloxy)-7-methoxy-chinazoline, 4-[(3-chloro-4-fluoro-phenyl)amino]-6-[cis-4-(N-methansulfonyl-N-methyl-amino)-cyclohexan-1-yloxy]-7-methoxy-chinazoline, 4-[(3-chloro-4-fluoro-phenyl)amino]-6-[cis-4-(N-acetyl-N-methyl-amino)-cyclohexan-1-yloxy]-7-methoxy-chinazoline, 4-[(3-chloro-4-fluoro-phenyl)amino]-6-(trans-4-methylamino-cyclohexan-1-yloxy)-7-methoxy-chinazoline, 4-[(3-chloro-4-fluoro-phenyl)amino]-6-[trans-4-(N-methansulfonyl-N-methyl-amino)-cyclohexan-1-yloxy]-7-methoxy-chinazoline, 4-[(3-chloro-4-fluoro-phenyl)amino]-6-(trans-4-dimethylamino-cyclohexan-1-yloxy)-7-methoxy-chinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-chinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-chinazoline, 4-[(3-chloro-4-fluoro-phenyl)amino]-6-(1-methansulfonyl-piperidin-4-yloxy)-7-methoxy-chinazoline, 4-[(3-chloro-4-fluoro-phenyl)amino]-6-(1-cyano-piperidin-4-yloxy)-7-methoxy-chinazoline, und 4-[(3-chloro-4-fluoro-phenyl)amino]-6-{1-[(2-methoxyethyl)carbonyl]-piperidin-4-yloxy}-7-methoxy-chinazoline, optionally in the form of the racemates, the enantiomers, the diastereomers and optionally the pharmacologically acceptable acid addition salts, solvates and/or the hydrates thereof.

Moreover, the compound could be from the group of betamimetika, antiallergika, derivates of ergotalcaloids, triptane, CGRP-antagonists, phosphodiesterase-V-inhibitores, optionally in the form of the racemates, the enantiomers, the diastereomers and optionally the pharmacologically acceptable acid addition salts and the hydrates thereof.

Preferably one of the two mentioned salts of salbutamol (the hydrochloride or the citrate of salbutamol) is used in combination with one or more additional pharmacologically active substances as cited above together with an environmentally safe hydrofluorocarbon (HFC) as a propellant, an organic compound as a co-solvent, and an either an inorganic acid or an organic acid, in the aerosol solution formulation.

More preferably ipratropium is used as a pharmacologically active substance in this combination

Suitable HFC propellants are those which, when mixed with the co-solvent(s), form a homogeneous propellant system in which a therapeutically effective amount of the medicament can be dissolved. The HFC propellant must be toxicologically safe and must have a vapor pressure which is suitable to enable the medicament to be administered via a pressurized MDI. Additionally, the HFC propellant must be compatible with the components of the MDI device (such as containers, valves, and sealing gaskets, etc.) which is employed to administer the medicament. Preferred HFC propellants are 1,1,1,2-tetrafluoroethane (HFC-134(a)) and 1,1,1,2,3,3,3,-heptafluoropropane (HFC-227). HFC-134(a) is particularly preferred. Other examples of HFC propellants are HFC-32 (difluoromethane), HFC-143(a) (1,1,1-trifluoroethane), HFC-134 (1,1,2,2-tetrafluoroethane), and HFC-152a (1,1-difluoroethane).

It will be apparent to those skilled in the art that non-halogenated hydrocarbon propellants may be used in place of the HFC propellants in the present invention. Examples of non-halogenated hydrocarbons are saturated hydrocarbons, including propane, n-butane, and isobutane, and ethers, including diethyl ether.

It will also be apparent to those skilled in the art that, although the use of a single HFC propellant is preferred, a mixture of two or more HFC propellants, or a mixture of at least one HFC propellant and one or more non-CFC propellants, may be employed in the aerosol solution formulation of the present invention.

The acid in the formulation provides stability against degradation or decomposition of the medicament resulting largely from interaction of the medicament with the co-solvent and/or water present in the solution formulation. The acid can be an inorganic or an organic acid.

Examples for an inorganic or mineral acid are hydrochloric acid, sulfuric acid, nitric acid, or phosphoric acid, or the like. More preferably, the inorganic acid is hydrochloric acid.

Alternatively the acid is selected from the group of acids known to those skilled in the art as organic acids, which are in most cases considered to be weak acids relative to the inorganic acids. Representative of this group and preferred in this invention are ascorbic acid, citric acid, lactic acid, malic acid, benzoic acid and tartaric acid. According to this invention, citric acid and ascorbic acid are the most preferred organic acids.

The formulations according to the invention are characterized in that the concentration of the acid is in a range that corresponds with a pH range of 2.0-6.0 in aqueous solution. In preferred aerosol solution formulations according to the invention the concentration of the acid is in a range that corresponds with a pH range of 2.5-5.0, more preferred 3.0-4.5 in aqueous solution.

The formulations according to the invention can be prepared in analogy to methods known in the art.

If desired, pharmaceutically acceptable excipients can be included in the aerosol solution formulations of the present invention. For example, a soluble surface active agent can be added in order to improve the performance of valve systems employed in the MDI devices used for the aerosol administration of the formulations. Examples of preferred surface active agents are sorbitan trioleate, lecithin, and isopropylmyristate. Other suitable lubricants are well known in the art (see, for example, Published European Patent Application No. 0372777 (EPO 893122705)). Other excipients are: (a) antioxidants, for example ascorbic acid and tocopherol; (b) taste masking agents, for example, menthol, sweeteners, and artificial or natural flavors; and (c) pressure modifying agents, for example, n-pentane, iso-pentane, neo-pentane and n-hexane.

The co-solvent preferably is an organic compound. Examples of co-solvents applicable within the formulations according to the invention are: alcohols, for example, ethyl alcohol, isopropyl alcohol, and benzyl alcohol; glycols for example, propylene glycol, polyethylene glycols, polypropylene glycols, glycol ethers, and block copolymers of oxyethylene and oxypropylene; and other substances, for example, glycerol, polyoxyethylene alcohols, polyoxtethylene fatty acid esters, and glycofurols (for example glycofurol 75).

Examples of co-solvents that may be inert to interaction with the medicament(s) are hydrocarbons, for example, n-propane, n-butane, isobutane, n-pentane, iso-pentane, neo-pentane, and n-hexane; and ethers, for example, diethyl ether.

A preferred co-solvent according to this invention is ethyl alcohol (ethanol).

The amount of co-solvent is preferably in the range of 5-50% (w/w) of the total composition. More preferably, the amount of co-solvent in the formulation according to the invention is in the range of 10-40% (w/w), preferably in the range of 15-30%.

Small amounts of water may be added to the formulation in order to enhance its solvency towards active substances and excipients. Preferably up to 5% (w/w) of water, more preferably up to 3%, and most preferably up to 2% of water is used in formulations containing water in addition to the co-solvent.

Another preferred embodiment of the invention is directed to formulations that do not contain any water. In these water-free formulations the amount of co-solvent is preferably in the range of about 20-60% (w/w), more preferably in the range of about 30-50% (w/w).

The formulations according to the invention can be administered with inhalers known in the art (Metered dose inhalers=MDIs), which contain multiple doses of a formulation.

In another aspect the invention is directed to the use of an aerosol solution formulation as described hereinbefore for the manufacture of a medicament for the treatment of respiratory complaints, particularly COPD (chronic obstructive pulmonary disease) and asthma.

In yet another aspect the invention is directed to a method for treatment of respiratory complaints, such as in particular COPD (chronic obstructive pulmonary disease) or asthma, characterized by the administration of an aerosol solution formulation as described hereinbefore.

In a further aspect the invention is directed to a pressurized, metered dose inhaler characterized in that the pharmaceutical formulation comprises an acid addition salbutamol salt, an HFC propellant, a co-solvent, and an inorganic or organic acid.

The following examples serve to illustrate the present invention further without restricting its scope to the embodiments provided hereinafter by way of example.

### I. FORMULATION EXAMPLES

| | |
|---|---|
| Component | Concentration [% w/w] |

A)

| | |
|---|---|
| Salbutamol citrate | 0.173 |
| Ethanol abs. (USP) | 30.000 |
| Water (purified, USP) | 2.000 |
| Citric acid (USP) | 0.004 |
| HFC-134a | 67.823 |

B)

| | |
|---|---|
| Salbutamol hydrochloride | 0.221 |
| Ethanol abs. (USP) | 30.000 |
| Water (purified, USP) | 2.000 |
| Citric acid (USP) | 0.003 |
| HFC-134a | 67.777 |

C)

| | |
|---|---|
| Salbutamol citrate | 0.173 |
| Ipratropium bromide | 0.021 |
| monohydrate | |
| Ethanol abs. (USP) | 30.000 |
| Water (purified, USP) | 2.000 |
| Citric acid (USP) | 0.004 |
| HFC-134a | 67.802 |

D)

| | |
|---|---|
| Salbutamol citrate | 0.221 |
| Ipratropium bromide | 0.040 |
| monohydrate | |
| Ethanol abs. (USP) | 30.000 |
| Water (purified, USP) | 2.000 |
| Citric acid (USP) | 0.003 |
| HFC-134a | 67.736 |

E)

| | |
|---|---|
| Salbutamol hydrochloride | 0.110 |
| Ethanol abs. (USP) | 40.000 |
| Water (purified, USP) | 2.000 |
| Citric acid (USP) | 0.003 |
| HFC-134a | 57.888 |

F)

| | |
|---|---|
| Salbutamol citrate | 0.173 |
| Ipratropium bromide | 0.021 |
| monohydrate | |
| Ethanol abs. (USP) | 40.000 |
| Citric acid (USP) | 0.004 |
| HFC-134a | 57.802 |

The aforementioned formulations can be prepared by conventional methods known in the state of the art.

### II. PREPARATION OF SALBUTAMOL HYDROCHLORIDE

Salbutamol base is added step wise to a 1:1 (w/w) mixture of ethanol and diethylether under stirring until a 30% w/w solution has been formed. The solution is cooled to approximately 10° C. Gaseous hydrochloric acid is then passed through the solution under continuous stirring and cooling to approximately 10° C. The total amount of gaseous hydrochloric acid passed through the solution should correspond to the 5-fold molar amount of salbutamol base. After the entire amount of hydrochloric acid gas has been introduced, the resulting suspension is cooled to approximately 0° C. The precipitated salbutamol hydrochloride is filtered off. The isolated residue is then recrystallised twice from ethanol.

The resulting salbutamol hydrochloride is a white crystalline powder. The melting point is

### III. PREPARATION OF SALBUTAMOL CITRATE

A 30% w/w solution of citric acid in water is added to a suspension of powdery salbutamol base in water under stirring at ambient temperature. After the salbutamol base has been completely dissolved, aceton is added to the solution under stirring at ambient temperature until the proportion of acetone amounts to ca. 80% w/w of the mixture. The mixture is then cooled to approximately 0° C. and the precipitated salbutamol citrate is filtered off. The isolated residue is the recrystallised twice from mixture of ethanol/ethyl acetate 70:30 (w/w).

The resulting salbutamol citrate appears in white crystals. It melts under decomposition at approximately 133° C. (onset determined by differential scanning calorimetry, heating rate 10° C./min).

We claim:

1. An aerosol solution formulation comprising an acid addition salt of salbutamol, an HFC propellant, a co-solvent, and an inorganic or an organic acid.

2. An aerosol solution formulation according to claim 1, wherein the formulation contains 0.001 to 1% salbutamol.

3. An aerosol solution formulation according to claim 1, wherein the acid addition salt of salbutamol is the hydrochloride of salbutamol or the citrate of salbutamol.

4. An aerosol solution formulation according to claim 1, wherein the formulation contains one or more additional pharmacologically active substances.

5. An aerosol solution formulation according to claim 4, wherein the additional pharmacologically active substance is selected from anticholinergics, beta-sympathomimetics, steroids, PDEIV-inhibitors, LTD4-Antagonists, EGFR-Kinase-Inhibitors.

6. An aerosol solution formulation according to claim 5, wherein the additional pharmacologically active substance is ipratropium bromide.

7. An aerosol solution formulation according to claim 1, wherein the formulation contains (a) salbutamol citrate or salbutamol hydrochloride, and (b) ipratropium bromide.

8. An aerosol solution formulation according to claim 1, wherein the HFC propellant is selected from HFC-134(a), HFC-227, HFC-32, HFC-143(a), HFC-134, HFC-152a, and mixtures thereof.

9. An aerosol solution formulation according to claim 1, wherein the acid is hydrochloric acid, sulfuric acid, nitric acid, or phosphoric acid.

10. An aerosol solution formulation according to one claim 1, wherein the acid is ascorbic acid, citric acid, lactic acid, malic acid, benzoic acid, or tartaric acid.

11. An aerosol solution formulation according to claim 1, further comprising water in an amount of up to about 5%.

12. An aerosol solution formulation according to claim 1, wherein the co-solvent is an organic compound.

13. An aerosol solution formulation according to claim 1, wherein the co-solvent is selected from alcohols, glycols, glycol ethers, block copolymers of oxyethylene and oxypropylene, glycerol, polyoxyethylene alcohols, polyoxtethylene fatty acid esters or glycofurols.

14. An aerosol solution formulation according to claim 1, wherein the co-solvent is present in an amount in the range of 5-50% (w/w).

15. An aerosol solution formulation according to claim 1, wherein said formulation does not contain water.

16. A method for treating a respiratory complaint in a subject, comprising administering to said subject by aerosol administration a therapeutically effective amount of a formulation according to claim 1.

17. A compound of formula Ia:

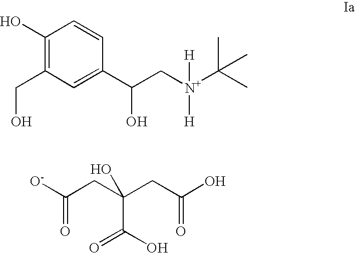

18. A compound of formula Ib:

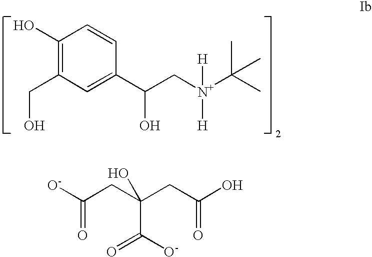

19. A compound of formula Ic:

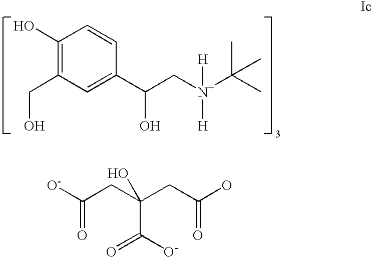

20. A pressurized metered dose inhaler comprising multiple doses of a formulation according to claim 1.

21. A pressurized metered dose inhaler according to claim 20, wherein the acid addition salt of salbutamol is the hydrochloride of salbutamol or the citrate of salbutamol.

22. A pressurized metered dose inhaler according to claim 20, wherein the formulation additionally contains ipratropium bromide

23. A pressurized metered dose inhaler according to claim 20, wherein the formulation contains (a) salbutamol citrate or salbutamol hydrochloride, and (b) ipratropium bromide.

24. An aerosol solution formulation according to claim 1, comprising salbutamol citrate or salbutamol hydrochloride, HFC-134a, ethanol, citric acid, and optionally water.

25. An aerosol solution formulation according to claim 24, further comprising ipratropium bromide monohydrate.

26. A pressurized metered dose inhaler comprising multiple doses of a formulation according to claim 24.
